(19) United States
(12) Patent Application Publication
(10) Pub. No.: US 2004/0068381 A1
(43) Pub. Date: Apr. 8, 2004

(54) Method of handling database for bioinformatics

(75) Inventors: Jai Hoon Kim, Yongin-si (KR); Min Jun Kim, Suwon-si (KR); Sung Jun Lee, Cheju-si (KR); Sung Hwa Lim, Bucheon-si (KR); Sang Min Park, Dangjin-gun (KR); Soo Jin Lee, Yongin-si (KR); Weon Tae Lee, Gwacheon-si (KR)

Correspondence Address: LAW OFFICES OF ROYAL W. CRAIG, Suite 153, 10 N. Calvert St., Baltimore, MD, 21202, US

(73) Assignees: DAEWOOD EDUCATIONAL FOUNDATION; Non-profit Organization.

(21) Appl. No.: 10/668,026

(22) Filed: Sep. 22, 2003

(30) Foreign Application Priority Data
Oct. 2, 2002 (KR) 2003-60295

(51) Int. Cl. G06F 19/00; G01N 33/48; G01N 33/50

(52) U.S. Cl. 702/20

(57) ABSTRACT

A method of handling a database for bioinformatics is disclosed. A server receives a sequence to be compared and analyzed from the user terminal to store it in a queue. When there exist other sequences to be compared and analyzed, the server reads the sequence of the current order from the database to compare it with all of sequences stored in the queue. That is, the server accesses the database once to use it for all of user requests currently being processed. Because the server accesses the database only once for each user request, the average system cost and response time are decreased. Furthermore, the threshold of the user request arrival rate that can be received on the same hardware in the present invention is higher compared to the convention method, so that larger amount of users can be provided the comparison/analysis service.

### CROSS-REFERENCE TO RELATED FOR APPLICATIONS

Pursuant to 35 U.S.C. 119(a) the present application derives priority from the following foreign filed patent application: Korean Patent Application No. 2003-60295, filed Oct. 2, 2002.

### BACKGROUND OF THE INVENTION

1. Field of the Invention

The present invention relates to a method for effectively handling a database used for Bioinformatics. Specifically, the present invention relates to a bioinformatics database handling method which does not wait for completion of processing of a previous user request being processed when another user makes a request for comparison of a bioinformatics-related sequence but simultaneously processes the new user request and the previous user request. Thus, the method can access the database only once for each user request so that system cost and response time can be decreased.

2. Description of the Background

Successful achievement of human gene projects performed in the early twenty-first century brought about rapid development in all life science fields. Due to completion of human gene map, studies on the human genes and the structures and functions of human proteins will be actively carried out in post genom. While a computer stores information represented by 0 and 1, the human genes stores information of about three billions represented by four letters, A, T, G and C. As the studies are performed, a vast amount of digital information is being accumulated and many databases related with bioinformatics, such as SwissProt, GenBank and EMBL, are opened to the public through a web.

There are various programs used for searching these bioinformatics databases for appropriate gene information at the request of a user. These programs are classified into a pattern match program such as FastA, Blast and Clustal W, which searches for data composed of A, T, G and C to perform sequence comparison, and a program of predicting a structure from data sequence, such as J-NET and J-PRED.

It is anticipated that future biologists will invest time in information analysis employing programs rather than in experiments. It means that the object of bioinformatics is not only to simply provide data but also to fully understand the gene itself. This is related with an increase in the demand for more powerful functions of programs and computing power. Furthermore, the quantity of data of databases used for bioinformatics is increased very rapidly as the studies on bioinformatics are executed. The increase in the capacity of databases makes efficient utilization of the databases in bioinformatics more important.

The programs such as FastA and Blast are provided to users through a web. A user connects to a server to transmit a protein sequence he/she wants to compare and analyze to the server. Then, the server reads sequences from the database and compares them with the protein sequence requested by the user. These programs operate based on a database. That is, the programs should access the database to read data and respond to a user's request for every user request. In case of FastA, for instance, a user transmits a sequence he/she wants to compare/analyze to FastA server. The transmitted sequence is compared with sequences stored in the database to check similarity, and sequences having similarity of higher than a predetermined value are returned to the user. Here, the server accesses the database for each user's request.

The cost required for providing the above-described service to a user through the aforementioned procedure will be explained hereinafter with reference to FIG. 1. C_{DB} denotes the cost required for accessing the database once for a user request, and C_{seq} represents the cost spent to compare all sequences read from the database with the sequence the user requests and analyze it. That is, the server spends the cost corresponding to C_{DB}+C_{seq} for one user request, Rn (n=1,2,3, . . . ). In this conventional structure, the server processes a user request immediately when there is no previous user request being processed. In the case where another user's request is being processed, however, newly generated other user requests are sequentially registered in a queue. In FIG. 1, a request R2 is registered in the queue because it was generated while a request R1 is being processed. The request R2 is processed when processing of R1 is completely finished and, at the same time, it is deleted from the queue.

When disc access time required for reading one block from the database is Cᵢₒ, the number of all sequences stored in the database is N_{b}, and the period of time required for comparing one protein sequence read from the database with the user-requested protein sequence, that is, processing time, is C_{cpu}, the server should bring all contents of the database to a memory whenever it compares one user-requested protein sequence with the sequences read from the database. The period of time required for this operation corresponds to the value obtained by multiplying the period of time consumed for accessing the database once by the number of all sequences stored in the database. When it is assumed that the time required for reading one block is uniform, access time C_{DB} can be represented as follows.

C_{DB}=Cᵢₒ·N_{b} (1)

C_{DB} represents the time required for accessing all sequences of the database, that is, disc access time for database search. The time consumed for comparison between sequences corresponds to the time C_{seq} required for comparing one user-requested sequence with the sequences read from the database. The period of time required for comparing all sequences of the database with the user-requested sequence can be represented as follows.

C_{seq}=C_{cpu}·N_{b} (2)

The average time taken for one user to connect to the server and compare one sequences with the sequences of the database, C_{avg}^{o}, which corresponds to the sum of the time of equation (a) and the time of equation (2), can be represented as follows.

C_{avg}^{o}=C_{DB}+C_{seq}=CᵢₒN_{b}+C_{cpu}N_{b}=(Cᵢₒ+C_{cpu})N_{b} (3)

Now, the response time in the conventional method will be explained hereinafter. It is assumed that a user request is Poisson process with generation rate λ. While the server is processing a user request, when another new user request generates, the new user request is registered in the queue. That is, user requests are registered in the queue in the order of generation and they are sequentially provided in the order of registration. When it is assumed that service costs for all of requests are identical, it becomes M/G/1 queuing model.

Service time 1/μ is identical to the time required for processing one user request. That is, service time 1/μ corresponds to the average cost, C_{avg}^{o}, for providing comparison/analysis service for one user request. Here, service rate μ is represented by

$$\frac{1}{{C}_{avg}^{o}} .$$

The result obtained by substituting the user request generation rate λ and service rate μ for the response time of M/G/1 queuing model is represented by the following equation (4).

$${W}_{o} = {( \frac{1}{{C}_{avg}^{o}} )}^{- 1} + \frac{λ \cdot {( \frac{1}{{C}_{avg}^{o}} )}^{- 2}}{2 ( 1 - \frac{λ}{1 / {C}_{avg}^{o}} )} = {C}_{avg}^{o} + \frac{λ {C}_{avg}^{o2}}{2 ( 1 - λ \cdot {C}_{avg}^{o} )} \qquad ( 4 )$$

As described above, the conventional method should search the database for each user request so that a vast amount of system cost is required. Furthermore, overload may be applied to the server to lengthen the response time.

### SUMMARY OF THE INVENTION

Accordingly, the present invention has been made in view of the above problems, and it is an object of the present invention is to provide a database handling method in which, when a user requests the database server to process comparison of a bioinformatics-related sequence, the server does not wait for completion of processing of a previous user request being processed but processes the new user request and the previous user request simultaneously, so that the server can access the database only once for each user request, thereby saving system cost and response time.

The present invention can be preferably implemented through a server that is associated with a database for storing sequence information related with bioinformatics and connected to each user terminal through a specific communication network. The server handles the database according to the present invention in order to compare a sequence requested from each user terminal with sequences of the database and analyze a result of comparison.

Specifically, the server receives a sequence to be compared and analyzed from the user terminal to store it in a queue in a first step. In a second step, the server checks whether or not there exist other sequences to be compared and analyzed in the queue, simultaneously with the first step. When there exist other sequences to be compared and analyzed, the server reads the sequence of the current order from the database to compare it with all of sequences stored in the queue in a third step. Then, the server judges whether or not there exists a sequence that has been compared and analyzed for all of sequences of the database among the sequences compared and analyzed at the third step, and removes the corresponding sequence from the queue in a fourth step. In a fifth step, the server increments the current order by one, while initializing the current order in the case where all of the sequences of the database have been read and returns to the second step.

### BRIEF DESCRIPTION OF THE DRAWINGS

The above and other objects, features and other advantages of the present invention will be more clearly understood from the following detailed description taken in conjunction with the accompanying drawings, in which:

FIG. 1 explains the service cost in the conventional method.

FIG. 2 illustrates an example of the configuration of a system to which the present invention is applied.

FIG. 3 is a flow chart showing an embodiment of the present invention.

FIG. 4 illustrates a process for explaining a detailed service method.

FIG. 5 illustrates a graph showing the comparison between system costs of the conventional method and the method of the present invention.

FIG. 6 illustrates a graph showing the comparison between the response time of the conventional method and that of the method according to the present invention.

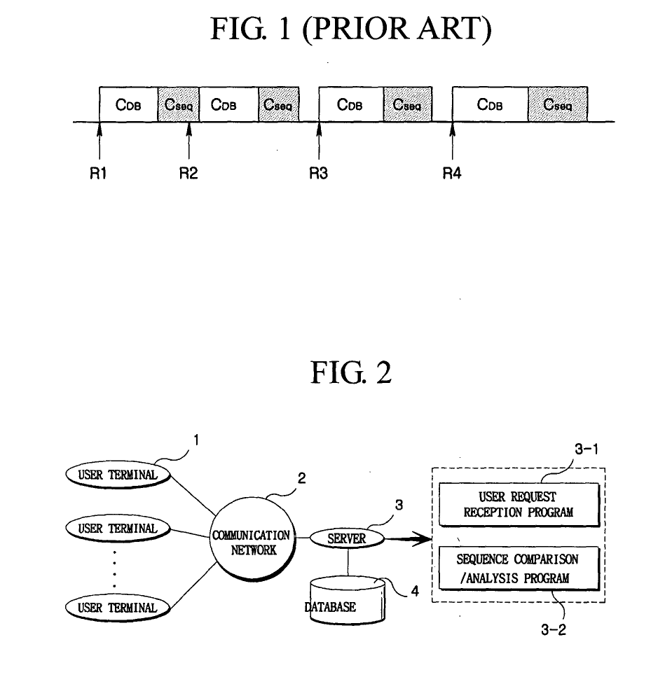

### DETAILED DESCRIPTION OF THE INVENTION

Reference will now be made in detail to the preferred embodiments of the present invention, examples of which are illustrated in the accompanying drawings.

The outline of a system to which the present invention is applied will be explained hereinafter with reference to FIG. 2. The subject of providing comparison/analysis service with respect to sequence information related with bioinformatics according to the present invention is a server 3. The server 3 is associated with a database 4 for storing bioinformatics-related sequence information and connected to each user terminal (client) 1 through a specific communication network 2. Here, the communication network 2 is preferably the Internet. The present invention can be preferably embodied according to a program 3-1 for receiving user requests and a program 3-2 for executing comparison/analysis of sequences, which are installed in the server 3.

Each user transmits sequence information that he/she wants to compare to the server 3 to request the server to carry out comparison/analysis of the sequence. The server 3 compares the user-requested sequence with sequence information stored in the database 4 to analyze a result of the comparison and sends the comparison/analysis result to the corresponding user terminal.

The core of the present invention is the method of accessing the database 4. The comparison/analysis method performed by the server 3 is identical to the conventional method so that detailed explanation therefore is omitted.

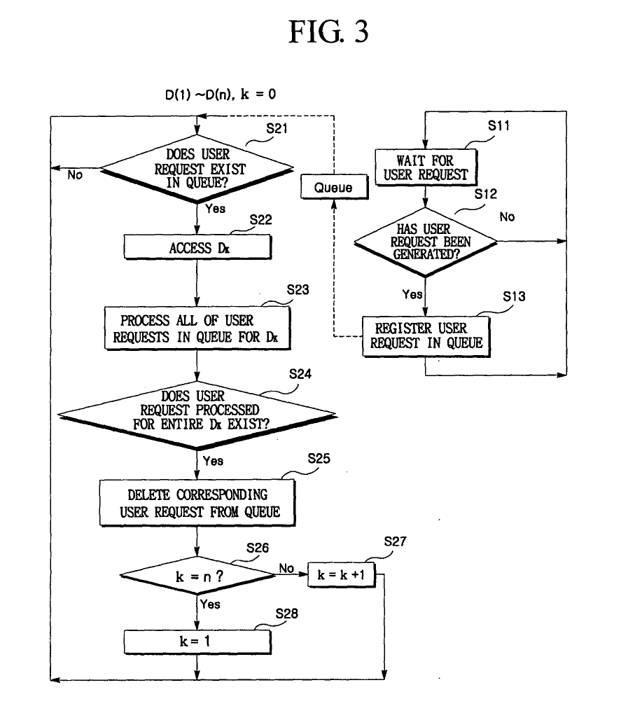

A preferred embodiment in the case where sequences stored in the database are D(n)(n=1,2,3, . . . ,n) is described with reference to FIG. 3.

In the first stage, the user request reception program 3-1 waits for a request from the user terminal 1 at step S11. When a user request is generated at step S12, the program 3-1 receives a sequence that the user requests and stores it in a queue at step S113.

In the second stage, the sequence comparison/analysis program 3-2 checks whether or not there exists a user-requested sequence in the queue at step S21 simultaneously with the first stage S11, S12 and S13. That is, the user request reception program 3-1 and sequence comparison/analysis program 3-2 operate simultaneously but they operate independently, exchanging sequences through the queue.

In the mean time, the sequence comparison/analysis program 3-2 initializes a specific parameter k to set in association with the operation thereof. At step S21, when there is a user-requested sequence in the queue, the sequence comparison/analysis program 3-2 reads the kth sequence from the database 4 at step S22, and then compares it with all of sequences stored in the queue at step S23 (Third stage).

In addition, the sequence comparison/analysis program 3-2 judges whether or not there is a user-requested sequence that has been compared/analyzed with respect to all sequences D(1) to D(n) of the database 4 among the sequences compared/analyzed in the third stage, at step S24. If there exists a user-requested sequence that has been compared/analyzed with respect to all sequences D(1) to D(n) of the database 4, the program 3-2 deletes it from the queue at step S25 (Fourth stage). That is, the sequence for which comparison is finished is eliminated from the queue. After the fourth stage, in the fifth stage including steps S26, S27 and S28, the program 3-2 increments k by one in case of k≠n but initializes k when k=n, and then returns to step S21.

As described above, the present invention processes currently requested sequence and the previous requested sequence being processed, simultaneously. This is possible because all of data of the database can be processed irrespective of data processing order since all of sequences of the database are generally searched in sequence search of bioinformatics and there is no dependence among data items of bioinformatics database.

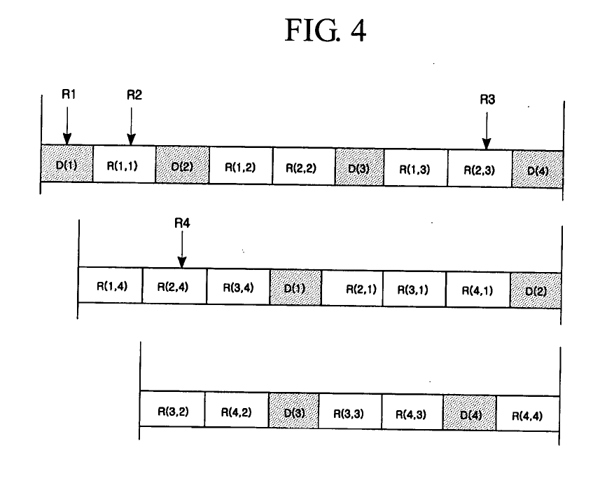

An embodiment where comparison/analysis service is provided for four user requests Rn(n=1,2,3,4) will be explained hereinafter with reference to FIG. 4. Here, D(i) denotes the cost needed to access the ith sequence of the database. It is assumed that the database has only four sequences. R(i,j) represents the cost required for comparing the ith user request with the jth database sequence.

The server reads the first sequence D(1) and processes service R(1,1) for a user request R1. When a user request R2 generates while the server is processing service R(1,1), the server reads the second sequence D(2) and processes service R(1,2) for R1 and service R(2,2) for R2.

That is, the server accesses the database only once to process multiple user requests so that the number of times of accessing the database can be reduced. The service for user request R1 is finished after the server reads up to the fourth sequence D(4). After the server reads up to the fourth sequence D4, because the server did not process the request R2 for the first sequence D1, it executes a routine of reading and processing the first sequence D1 together with a user request R3. That is, although the user requests are processed until all of sequences are accessed, there is an advantage that processing of a new request is not delayed until processing for the previous user request being processed is finished. This is possible because the order of reading data from the database does not affect the result of sequence comparison in bioinformatics.

The cost required for providing the comparison/analysis service according to the present invention can be obtained based on the processing time taken to start access from the start point of the database to return to the start point. Let it be assumed that user request generation rate is λ, and the sum of the database access time required for accessing the overall database and a period of time consumed for comparing an arrived user request with the database is

$${C}_{rotal}^{cp} .$$

The average number of requests generated during this period,

$${C}_{total}^{cp} ,$$

becomes

$$λ \cdot {C}_{total}^{cp} .$$

During the period

$${C}_{total}^{cp} ,$$

the database is accessed only once, and the total cost needed during the period is obtained as follows.

$${C}_{total}^{cp} = {C}_{DB} + λ \cdot {C}_{total}^{cp} \cdot {C}_{seq} \qquad ( 5 )$$

The equation (5) is rearranged for

$${C}_{total}^{cp}$$

as follows.

$${C}_{total}^{cp} = \frac{{C}_{DB}}{1 - λ \cdot {C}_{seq}} \qquad ( 6 )$$

In consideration of the case that no request is generated, the system cost required for processing one user request can be obtained as follows.

$${C}_{avg}^{cp} = \frac{{C}_{DB}}{{C}_{total}^{cp} \cdot λ} ( 1 - {ⅇ}^{- λ \frac{{C}_{DB}}{1 - λ {C}_{seq}}} ) + {C}_{seq} \qquad ( 7 )$$

The equation (7) calculates the cost required for processing one user request in consideration of the value obtained by dividing the equation (5) by the number of user requests, that is, probability of generation of user requests for one period,

$$1 - {ⅇ}^{- {λC}_{total}^{cp}} .$$

The equation (7) can be rearranged as follows.

$${C}_{avg}^{cp} = ( \frac{1}{λ} - {C}_{seq} ) ( 1 - {ⅇ}^{- λ \frac{{C}_{DB}}{1 - λ {C}_{seq}}} ) + {C}_{seq} \qquad ( 8 )$$

The response time when the comparison/analysis service is provided according to the present invention is obtained as follows.

The point of time when the service is completed for one user request corresponds to the time at which all of sequences of the database are read and processing for the read sequences is finished. Each of read sequences of the database is also used for other user requests processed simultaneously. That is, the response time in the present invention is identical to the sum of the period of time required for processing one user request and the period of time, (λ·W_{cp}·C_{seq}), taken to process another user request processed simultaneously during the period of time, (C_{DB}+C_{seq}), which is represented by the following equation (9).

W_{cp}=C_{DB}+C_{seq}+λ·W_{cp}C_{seq} (9)

The equation (9) is rearranged as follows.

$${W}_{cp} = \frac{{C}_{DB} + {C}_{seq}}{1 - λ \cdot {C}_{seq}} \qquad ( 10 )$$

The performance of the conventional method is compared with that of the method of the present invention below.

Each of the conventional method and the method of the invention has a threshold for user request arrival rate λ. The threshold of the arrival rate λ represents the maximum arrival rate that satisfies the condition that a server utilization rate is smaller than 1. The server utilization rate can be represented by the value obtained by multiplying the user request arrival rate by the average cost required for the server to process one user request. When the server utilization rate is smaller than 1, the comparison/analysis service can be provided. The server utilization rate can be increased higher than 1 by using a technique capable of accessing the database and using a CPU, simultaneously.

Letting it be assumed that sequence comparison is sequentially carried, the thresholds in the conventional method and the method of the present invention are obtained as follows.

1) Conventional Method

The average cost required for processing one user request in the conventional method can be represented by the equation (3), which is expressed as λ·C_{avg}^{o}<1. That is, the threshold of λ can be represented as follows.

$$λ \leq \frac{1}{{C}_{seq} + {C}_{DB}} \qquad ( 11 )$$

2) Method of the Invention

According to the present invention, the server utilization rate becomes

$$λ \cdot \frac{1}{λ}$$

so that it satisfies the condition all the time. Furthermore, in the equation (9), Cₜₒₜₐₗcp is a positive number ad C_{DB} is a negative number. That is, 1−λ·C_{seq}>0 should be satisfied. This is solved to obtain the threshold of λ as follows.

$$λ ≺ \frac{1}{{C}_{seq}} \qquad ( 12 )$$

The database GenBank (Protein Sequence Database of Rip International Release 72.02), actually being used in bioinformatics, was managed by Ros Alamos research institute in support of National Institute of Health in 1981, and transferred to National Center for Biotechnology Information (NCBI) under the control of National Library of Medicine in 1992. With this GenBank, cytochrome that acts upon oxidization and reduction of cells, one of human proteins, was used as a user-requested sequence. As a result, C_{DB} was 3.99 sec and C_{seq}, the cost required for comparing the user-requested sequence with all of sequences of the database, was 19.98 sec.

In comparison of the equation (11) to the equation (12), the maximum value of λ in the equation (12) is larger than that of λ in the equation (11) all the time. That is, with hardware having the same performance, the method of the invention can receive larger number of users than the conventional method.

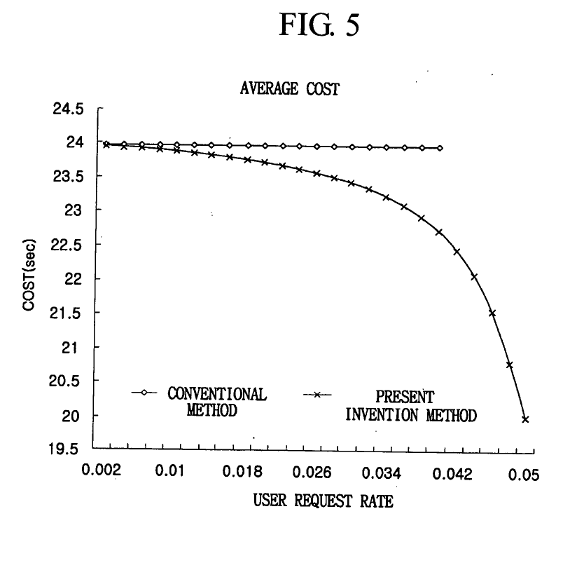

The system cost in case of the method of the present invention is compared with the system cost of the convention method using the equations (3) and (8) with reference to FIG. 5. The graph of FIG. 5 represents values up to thresholds for both of the methods. In FIG. 5, x-axis denotes the user request rate λ and y-axis indicates the system cost.

With the method of the present invention, the system cost decreases as λ increases. The cost per user is rapidly reduced because the database access cost decreases as the user request rate λ increases.

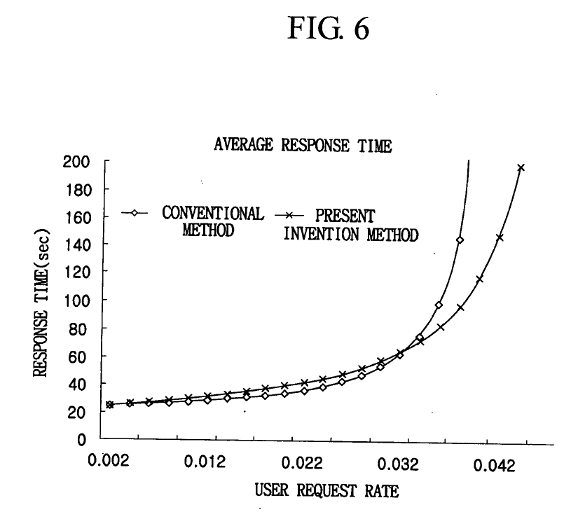

The response time of the method according to the present invention is compared with that of the convention method with reference to FIG. 6. In FIG. 6, x-axis denotes the user request rate λ while y-axis indicates the average response time of program for an arbitrary user request. Referring to FIG. 6, while the response time abruptly increases as it reaches the threshold in the conventional method, the method of the invention shows shorter response time. This is because the server reads the database immediately read in case of a small number of users. Furthermore, the response time of the present invention is shorter than that of the conventional method because the number of times of accessing the database in the present invention is smaller than that of the conventional method.

As described above through FIG. 3, the database handling method according to the present invention can be embodied by the programs executed in the server 3. Accordingly, the present invention can be applied to a recording medium in which a computer program capable of executing the first to fifth stages is recorded.

According to the present invention, the server accesses the database only once in order to handle all of user requests currently being processed. Accordingly, the average system cost is reduced and satisfactory response time is obtained. Moreover, the threshold of the user request arrival rate that can be received on the same hardware in the present invention is higher compared to the convention method, so that larger amount of users can be provided the comparison/analysis service.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

We claim:

1. A method for handling a database for bioinformatics, in which a server, which is associated with a database for storing sequence information related with bioinformatics and connected to each user terminal through a specific communication network, compares a sequence requested from each user terminal with sequences of the database to analyze a result of the comparison, the method comprises:
(a) a first step of receiving the sequence from the user terminal to store it in a queue;
(b) a second step of checking whether or not there exist other sequences to be compared and analyzed in the queue, simultaneously with the first step;
(c) a third step of reading the sequence of the current order from the database to compare it with all of sequences stored in the queue when there exist other sequences to be compared and analyzed at the second step;
(d) a fourth step of judging whether or not there exists a sequence that has been compared and analyzed for all of sequences of the database among the sequences compared and analyzed at the third step, and removing the corresponding sequence from the queue; and,
(e) a fifth step of incrementing the current order by one, initializing the current order when all of the sequences of the database have been read and returning to the second step.

2. A recording medium readable by a computer, in which a computer program for executing the first to fifth steps according to claim 1 is recorded.
